(19) United States
(12) Patent Application Publication
(10) Pub. No.: US 2015/0323548 A1
(43) Pub. Date: Nov. 12, 2015

(54) METHODS OF MONITORING THE MODULATION OF THE KINASE ACTIVITY OF FIBROBLAST GROWTH FACTOR RECEPTOR AND USES OF SAID METHODS

(71) Applicants: Diana Graus Porta, Basel (CH); Vito Guagnano, Basel (CH); Estelle Marrer, Basel (CH); Pablo Verdes, Oxford (GB)

(72) Inventors: Diana Graus Porta, Basel (CH); Vito Guagnano, Basel (CH); Estelle Marrer, Basel (CH); Pablo Verdes, Oxford (GB)

(73) Assignee: Novartis AG, Basel (CH)

(21) Appl. No.: 14/804,491

(22) Filed: Jul. 21, 2015

Related U.S. Application Data
(63) Continuation of application No. 12/989,841, filed on Oct. 27, 2010.

(30) Foreign Application Priority Data
Apr. 29, 2008 (EP) 08155401.6
May 23, 2008 (EP) 08156856.0

(51) Int. Cl. G01N 33/68; A61K 31/496; A61K 31/506

(52) U.S. Cl. CPC G01N 33/6872; A61K 31/506; A61K 31/496; G01N 2333/71

(57) ABSTRACT

The present invention relates generally to methods of in vitro diagnostics, in particular the use of a compound selected from the group consisting of fibroblast growth factor 23 (FGF23), inorganic phosphorus (P), the product of inorganic phosphorus and total calcium (P×tCa), osteopontin (OPN) and parathyroid hormone (PTH) as biomarker. Said biomarkers can be used to monitor the modulation of fibroblast growth factor receptor (FGFR) kinase activity, in particular its inhibition, and/or the occurrence of secondary effects of FGFR inhibition. The invention further provides methods and kits relating to these uses.

### FIELD OF THE INVENTION

The present invention relates generally to methods of in vitro diagnostics, in particular the use of a compound selected from the group consisting of fibroblast growth factor 23 (FGF23), inorganic phosphorus (P), the product of inorganic phosphorus and total calcium (P×tCa), osteopontin (OPN) and parathyroid hormone (PTH) as biomarker. Said biomarkers can be used to monitor the modulation of fibroblast growth factor receptors (FGFRs) kinase activity, in particular its inhibition, and/or the occurrence of secondary effects of FGFR inhibition.

### BACKGROUND OF THE INVENTION

The fibroblast growth factor (FGF) family and their signaling receptors are associated with multiple biological activities (proliferation, survival, apoptosis, differentiation, motility) that govern key processes (development, angiogenesis, metabolism) for the growth and maintenance of organisms from worms to humans. 22 distinct FGFs have been identified, all sharing a conserved 120-aminoacids core domain with 15-65% sequence identity. FGFs mediate their cellular responses by binding to and activating a family of four RTKs FGFR1 to FGFR4, all of them existing in several isoforms (Lee P L et al., Science 245: 57-60 (1989); Givol D et al., FASEB J. 6:3362-9 (1992); Jaye M et al., EMBO J. 7:963-9 (1988); Omitz D M & Itoh N, Genome Biol. 2 (2001)). Ligand binding induces receptor dimerization events and activation of the kinase leading to phosphorylation and/or recruitment of downstream molecules and activation of intracellular signaling pathways.

The biological roles of FGFs/FGFRs have been investigated by analysis in specific developmental systems, expression patterns and gene targeting approaches in mouse models. These studies have demonstrated their involvement in many biological functions including angiogenesis and wound healing, development and metabolism. A variety of human craniosynostosis syndromes and skeletal dysplasias have been linked to specific gain of function mutations in FGFR1, FGFR2 and FGFR3 that lead to severe impairment in cranial, digital and skeletal development. Webster M K & Donoghue D J, Trends Genet. 1997 13:178-82 (1997); Wilkie A O, Hum. Mol. Genet. 6:1647-56 (1997).

Epidemiological studies have reported genetic alterations and/or abnormal expression of FGFs/FGFRs in human cancers: translocation and fusion of FGFR1 to other genes resulting in constitutive activation of FGFR1 kinase is responsible for 8p11 myeloproliferative disorder (MacDonald D & Cross N C, Pathobiology 74:81-8 (2007)). Recurrent chromosomal translocations of 14q32 into the immunoglobuling heavy chain switch region result in deregulated over-expression of FGFR3 in multiple myeloma (Chesi M et al., Nature Genetics 16:260-264 (1997); Chesi M et al., Blood 97:729-736 (2001)). Gene amplification and protein over-expression have been reported for FGFR1, FGFR2 and FGFR4 in breast tumors (Adnane J et al., Oncogene 6:659-63 (1991); Jaakkola S et al., Int. J. Cancer 54:378-82 (1993); Penault-Llorca F et al., Int. J. Cancer 61: 170-6 (1995); Reis-Filho J S et al., Clin. Cancer Res. 12:6652-62 (2006)). Somatic activating mutations of FGFR2 are known in gastric (Jang J H et al., Cancer Res. 61:3541-3 (2001)) and endometrial cancers (Pollock P M et al., Oncogene (May 21, 2007)) and somatic mutations in specific domains of FGFR3 leading to ligand-independent constitutive activation of the receptor have been identified in urinary bladder carcinomas (Cappellen D et al., Nature Genetics 23:18-20 (1999); Billerey C et al., Am. J. Pathol. 158(6):1955-9 (2001)). In addition, overexpression of FGFR3, mRNA and protein, has been found in this cancer type (Gomez-Roman J J et al., Clin. Cancer Res. 11(2 Pt 1):459-65 (2005)).

Thus, a compound capable of inhibiting the kinase activity of FGFRs is a likely candidate for the treatment of human cancers with deregulated FGFR signaling.

The utility of small molecular mass inhibitors of FGFR tyrosine kinase has already been validated (see Brown, A. P et al. (2005), Toxicol. Pathol. 33, p. 449-455; Xin, X. et al. (2006), Clin. Cancer Res., Vol 12(16), p. 4908-4915; Trudel, S. et al. (2005), Blood, Vol. 105(7), p. 2941-2948).

However, the determination of the therapeutic efficacy of such inhibitors in animal models is rather cumbersome as it involves for example measurement of tumor growth, the inhibition of auto-phosphorylation of FGF receptors and/or the phosphorylation of downstream molecules of the signaling cascade, such as Erk1/2. Albeit these methods are suitable in a pre-clinical setting, for clinical studies, a non-invasive method for determining the therapeutic efficacy in a simple and straight-forward manner is desirable.

Furthermore, nonclinical toxicity studies in rats and dogs with the FGFR tyrosine kinase inhibitor PD 176067 produced soft tissue mineralization. Due to the occurrence of this unwanted effect, it is concluded that further studies were necessary to determine whether said agent has the potential to be used for the treatment of cancer (see Brown, A. P et al. (2005), Taxicol. Pathol. 33, p. 449-455).

Ectopic mineralization, the inappropriate deposition of calcium phosphate salts in soft tissues and vascular system, can lead to morbidity and mortality (London G M et al., Curr. Opin Nephrol. Hypertens. 2005, 14:525-531).

Hence, there is a need in the art for biomarkers, reliable methods and corresponding kits useful for indicating the therapeutic efficacy of FGFR inhibitors. Furthermore, a method for the prediction of unwanted secondary effects following the administration of FGFR inhibitors, in particular of ectopic mineralization, would be of great use.

### SUMMARY OF THE INVENTION

It has surprisingly been found that compounds selected from the group consisting of fibroblast growth factor 23 (FGF23), inorganic phosphorus (P), the product of inorganic phosphorus and total calcium (P×tCa), osteopontin (OPN) and parathyroid hormone (PTH) are useful biomarkers which allow for the monitoring of the activity of fibroblast growth factor receptor (FGFR) inhibitors and may furthermore be useful in predicting the occurrence of secondary effects of FGFR inhibition, in particular of ectopic mineralization.

In particular, the present invention provides the use of FGF23 as a biomarker. Upon inhibition of FGFRs, anti-tumoral activity is found which is also translated into an increase of FGF23. The extent of the FGF23 increase correlates to the doses of the inhibitor used. At certain doses, secondary effects, in particular soft tissue and vascular mineralization, are detected. Due to this double connotation FGF23 may be regarded as a pharmacodynamic marker of FGFR inhibitors. The identification and validation of pharmacodynamic biomarkers that allow monitoring the biological activity of a drug is useful for dose selection and therapy optimization.

Furthermore, an overall analysis of potential biomarkers to predict and monitor the ectopic mineralization following Fibroblast Growth Factor Receptor modulation shows that compounds selected from the group consisting of FGF23, P, P×tCa, OPN and PTH are confirmed to be predictive markers of ectopic mineralization.

Accordingly, the invention provides in a first aspect for the use of a compound selected from the group consisting of FGF23, P, P×tCa, OPN and PTH as a biomarker, in particular for the modulation of kinase activity of FGFRs.

In one embodiment said compound is used to monitor the inhibition of fibroblast growth factor receptor kinase activity. Preferably, the compound is FGF23.

The invention further provides the use of a compound selected from the group consisting of fibroblast growth factor 23 (FGF23), inorganic phosphorus (P), the product of inorganic phosphorus and total calcium (P×tCa), osteopontin (OPN) and parathyroid hormone (PTH) as a safety marker for the prevention of secondary effects, in particular of ectopic mineralization. Preferably, said compound is FGF23.

In another aspect, the invention provides a method for determining the modulation of kinase activity of FGFR, in particular the inhibition of kinase activity, comprising the steps of
a) administering a FGFR inhibitor to a subject;
b) providing a sample of said subject;
c) determining the level of FGF23 of said sample; and
d) comparing said level of FGF23 of said sample with a reference level, wherein the reference level is the level of FGF23 in the subject before the onset of treatment with a FGFR inhibitor.

Further, a method for determining therapeutic efficacy of a FGFR inhibitor is provided, which comprises steps a) to d) of the above method, wherein the reference level is the level of FGF23 in the subject before the onset of treatment with a FGFR inhibitor.

Moreover, a method for determining one or more secondary effects of a FGFR inhibitor is provided comprising steps a) to d) of the above method, wherein the reference level is the level of FGF23 in the subject before the onset of treatment with a FGFR inhibitor.

The methods disclosed herein can be similarly performed with any one of the compound selected from the group consisting of P, P×tCa, OPN and PTH.

The invention is particularly useful in a clinical setting for dose selection, schedule selection, patient selection and therapy optimization.

The present invention will be described in more detail below. It is understood that the various embodiments, preferences and ranges may be combined at will. Further, depending on the specific embodiment, selected definitions, embodiments or ranges may not apply.

### BRIEF DESCRIPTION OF THE DRAWINGS

FIG. 1 is a graph showing the change of tumor volume in [mm³] during treatment with COMPOUND A of female athymic nude mice bearing NIH3T3/FGFR3^{S249C} subcutaneous tumors. White circles: COMPOUND A 0 mg/kg, qd, p.o.; black circles: COMPOUND A 10 mg/kg, qd, p.o.; grey circles: COMPOUND A 30 mg/kg, qd, p.o.; black triangles: COMPOUND A 50 mg/kg, qd, p.o.

FIG. 2 is a photograph showing the ex vivo analysis of tumors. Tumors were dissected 2 h after the last compound administration. Tumor tissue was lysed and FGFR3 was immunoprecipitated with a specific antibody. Immunocomplexes were resolved by SDS-PAGE, blotted onto PVDF membranes and probed with anti-pTyr antibody to monitor FGFR3 Tyr-phosphorylation. Membranes were stripped and reprobed with anti-FGFR3 antibody to monitor total FGFR3 protein levels.

FIG. 3 is a graph showing the change of tumor volume in [mm³] during treatment with COMPOUND A of female athymic nude mice bearing RT112/luciferasel subcutaneous xenografts. White circles: Vehicle 10 mg/kg, qd, p.o.; white squares: COMPOUND A 50 mg/kg, qd, p.o.; black triangles: COMPOUND A 75 mg/kg, qd, p.o.

FIG. 4 is a bar graph showing FGF23 levels in plasma samples recovered 2 h after the last administration of COMPOUND A or vehicle control at the indicated doses and schedule for 14 days (n=6) to female athymic mice bearing RT112/luciferasel subcutaneous xenografts. FGF23 levels were monitored using the FGF23 ELISA kit from Kainos, catalogue number CY-4000, and are expressed in pg/mL. Data are presented as means±SD.

FIG. 5 is scatter plot of the levels of inorganic phosphorus (P) [mg/dl], as described in example 2.

FIG. 6 is scatter plot of the serum levels of total calcium (tCa) [mg/dl].

FIG. 7 is scatter plot of the serum levels of P×tCa product [mg²/dl²].

FIG. 8 is scatter plot of the FGF23 serum levels [pg/ml].

FIG. 9 is a bar graph showing FGF23 levels in plasma samples from melanoma patients at pre-treatment or treated orally with TKI258 at 200, 300, 400 or 500 mg/day on a once daily continuous dose at cycle 1 day 15 and at cycle 1 day 26. FGF23 levels were monitored using the FGF23 ELISA kit from Kainos, catalogue number CY-4000, and are expressed in pg/mL. Data are presented as means±SD.

FIG. 10 shows a photograph of a tumor biopsy from a melanoma patient treated with 400 mg of TKI258 at cycle 1 Day 15, analyzed by immunohistochemistry with an antibody that recognizes phosphorylated and activated FGFR.

FIG. 11 is a graph showing the levels of FGF23 in 8 different renal cell carcinoma patients at baseline (C1D1) and upon treatment with 500 mg TK1258 at C1D15 and at C1D26, expressed as fold induction over baseline, this one being indicated as 1.

FIG. 12 is a photograph showing the ex vivo analysis of RT112 tumor xenografts. Tumors were dissected 3 h after compound administration. Tumor tissue was lysed and FRS2 tyrosine phosphorylation levels were analysed by western blot using an antibody from Cell Signaling (#3864) that detects FRS2 when phosphorylated on Tyr196. As a loading control, membrane was probed with an antibody from Sigma (#T4026) that detects b-tubulin.

FIG. 13 is a bar graph showing FGF23 levels in serum samples from rats treated with the indicated oral doses of TKI258 and obtained by sublingual bleeding 24 h after treatment with TKI258 or vehicle control. FGF23 levels were monitored using the FGF23 ELISA kit from Kainos, catalogue number CY-4000, and are expressed in pg/mL. Data are presented as means of n=4, ±SD. Data were compared by one-way Anova post-hoc Dunnett's versus vehicle.

FIG. 14 is a bar graph showing FGF23 levels in serum samples from rats treated with the indicated oral doses of the indicated compounds and obtained by sublingual bleeding 24 h after compound administration. FGF23 levels were monitored using the FGF23 ELISA kit from Kainos, catalogue number CY-4000, and are expressed in pg/mL. Data are presented as means of n=6, ±SD.

### DETAILED DESCRIPTION OF THE INVENTION

In a first aspect, the invention provides for the use of a compound selected from the group consisting of fibroblast growth factor 23 (FGF23), inorganic phosphorus (P), the product of inorganic phosphorus and total calcium (P×tCa), osteopontin (OPN) and parathyroid hormone (PTH) as a biomarker, in particular as a biomarker for the modulation, preferably inhibition of kinase activity of fibroblast growth factor receptor (FGFR). Said compound is preferably FGF23.

The fibroblast growth factor 23 (FGF23) is known. It is considered a member of the fibroblast growth factor family with broad biological activities. The sequence of the protein and/or the coding sequence of the protein can be retrieved from publicly available databases known in the art. Human FGF23 is also known in the art as ADHR; HYPF; HPDR2; PHPTC. Methods for determination are known in the field and are particularly described below.

The term “inorganic phosphorus” (P) is known in the filed and in particular refers to the blood level of inorganic phosphorus and may e.g. be measured in serum by ultraviolet method using kits for example from RANDOX Laboratories LTD, UK, and a clinical chemistry analyzer such as the HITACHI 717 analyzer (Roche Diagnostics).

The term “total calcium” (tCa) is known in the filed and in particular refers to the blood level of total calcium and may e.g. be measured in serum by ultraviolet method using kits for example from RANDOX Laboratories LTD and a clinical chemistry analyzer such as the HITACHI 717 analyzer.

The term “product of inorganic phosphorus and total calcium” (P×tCa) is known in the filed and in particular is obtained by multiplying the value levels of inorganic phosphorus (P) by the value levels of total calcium (tCa) in mg/dL.

Osteopontin (OPN) also referred to as secreted phosphoprotein 1, bone sialoprotein I or early T-lymphocyte activation 1, is known. It is considered an extracellular structural protein. Human osteopontin is known in the art as SPP1. Osteopontin may e.g. be measured using a kit such as the Osteopontin (rat) EIA Kit of Assay Designs, Inc., USA, following the manufacturer instructions.

Parathyroid hormone (PTH) or parathonnmone is known. It is considered a hormone involved in the regulation of the calcium level in blood. PTH may e.g. be determined using a solid phase radioimmunoessay such as the one available from Immutopics, Inc., USA.

In particular, the inhibition of FGFRs can be evaluated by determining the levels of one or more of the above mentioned compounds, preferably of FGF23, in a sample. Thereby, therapeutic efficacy of a FGFR inhibitor can be assessed.

The term “fibroblast growth factor receptor inhibitor” or “FGFR inhibitor” as used herein refers to molecules being able to block the kinase activity of fibroblast growth factor receptors. These may be macromolecules, such as antibodies, or small molecular mass compounds.

[01] In a preferred embodiment of the use and methods disclosed herein, the FGFR inhibitor is a small molecular mass compound. Examples of small molecular mass FGFR inhibitors include, but are not limited to, PD176067, PD173074, COMPOUND A, TKI258, or COMPOUND B. PD176067 (see Brown, C L et al., (2005), Toxicol. Pathol, Vol 33, p. 449-455. PD173074 is an FGF-R inhibitor from Parke Davis (see Mohammadi et al., EMBO J. 17: 5896-5904), of which specificity and potency are confirmed. It has the formula:

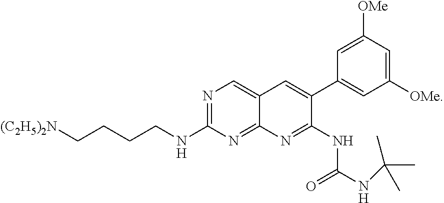

TKI258 was previously known as CHIR258 and is disclosed in WO02/22598 in example 109, as well as in Xin, X. et al., (2006), Clin. Cancer Res., Vol 12(16), p. 4908-4915; Trudel, S. et al., (2005), Blood Vol. 105(7), p. 2941-2948). COMPOUND A is a pan-FGFR inhibitor, e.g. disclosed in WO 06/000420 in example 145 as 3-(2,6-Dichloro-3,5-dimethoxy-phenyl)-1-{6-[4-(4-ethyl-perpazin-1-yl)-phenylamino]-pyrimidin-4-yl}-1-methyl urea. COMPOUND B is a derivative of [4,5′]bipyrimidinyl-6,4′-diamine. Its structure is described in WO 08/008747 (compound number 4 in table 1). The compounds may be prepared as disclosed or by analogy to the procedures described in these references.

In a preferred embodiment of the methods and use disclosed herein, the FGFR inhibitor is COMPOUND A in the free base or a suitable salt form.

“Therapeutic efficacy” as used herein refers to the treatment, prevention or delay of progression of human malignancies or conditions, such as proliferative diseases and non-cancer disorders. In case of proliferative diseases, therapeutic efficacy refers e.g. to the ability of a compound to reduce the size of a tumor or stop the growth of a tumor.

The disease may be, without being limited to, a benign or malignant proliferative disease, e.g. a cancer, e. g. tumors and/or metastasis (wherever located). In a preferred embodiment, the proliferative disease of the methods of the present invention is a cancer. Preferably said cancer is caused or related to deregulated FGFR signalling.

The proliferative diseases include, without being limited to, cancers of the bladder, cervix, or oral squamous cell carcinomas with mutated FGFR3 and/or elevated FGFR3 expression (Cappellen et al., Nature Genetics 1999, 23; 19-20; van Rhijn et al., Cancer Research 2001, 61: 1265-1268; Billerey et al., Am. J. Pathol. 2001, 158:1955-1959, Gomez-Roman et al., Clin. Can. Res. 2005, 11:459-465; Tomlinson et al., J. Pathol. 2007 213:91-8; WO 2004/085676), multiple myeloma with t(4,14) chromosomal translocation (Chesi et al., Nature Genetics 1997, 16: 260-264; Richelda et al., Blood 1997, 90:4061-4070; Sibley et al., BJH 2002, 118: 514-520; Santra et al., Blood2003, 101: 2374-2476), breast cancers with gene amplification and/or protein overexpression of FGFR1, FGFR2 or FGFR4 (Elbauomi Elsheikh et al., Breast Cancer Research 2007, 9(2); Penault-Llorca et al., Int J Cancer 1995; Theillet et al., Genes Chrom. Cancer 1993; Adnane et al., Oncogene 1991; Jaakola et al., Int J Cancer 1993), endometrial cancer with FGFR2 mutations (Pollock, Oncogene 2007, 1-5), hepatocellular cancer with elevated expression of FGFR3 or FGFR4 or FGF ligands (Tsou, Genomics 1998, 50:331-40; Hu et al., Carcinogenesis 1996, 17:931-8; Qui. World J. Gastroenterol. 2005, 11:5266-72; Hu et al., Cancer Letters 2007, 252:36-42), any cancer type with an amplification of the 11 q13 amplicon, which contains the FGF3, FGF4 and FGF19 loci, for example breast cancer, hepatocellular cancer (Berns E M et al., Gene 1995, 159:11-8, Hu et al., Cancer Letters 2007, 252:36-42), EMS myeloproliferative disorders with abnormal FGFR1 fusion proteins (MacDonald, Cross Pathobiology 2007, 74:81-88), lymphomas with abnormal FGFR3 fusion proteins (Yagasaki et al., Cancer Res. 2001, 61:8371-4), glioblastomas with FGFR1 abnormal expression or mutations (Yamaguchi et al., PNAS 1994, 91:484-488; Yamada et al., Glia 1999, 28:66-76), gastric carcinomas with FGFR2 mutations or overexpression or FGFR3 mutations (Nakamura et al., Gastroentoerology 2006, 131:1530-1541; Takeda et al., Clin. Can. Res. 2007, 13:3051-7; Jang et al., Cancer Res. 2001, 61:3541-3), pancreatic carcinomas with abnormal FGFR1 or FGFR4 expression (Kobrin et al., Cancer Research 1993; Yamanaka et al., Cancer Research 1993; Shah et al., Oncogene 2002), prostate carcinomas with abnormal expression of FGFR1, FGFR4, or FGF ligands (Giri et al., Clin. Cancer Res. 1999; Dorkin et al., Oncogene 1999, 182755-61; Valve et al., Lab. Invest. 2001, 81:815-26; Wang, Clin. Cancer Res. 2004, 10:6169-78); pituitary tumors with abnormal FGFR4 (Abbas et al., J. Clin. Endocrinol. Metab. 1997, 82:1160-6), and any cancer that requires angiogenesis since FGFs/FGFRs are also involved in angiogenesis (see e.g. Presta et al., Cytokine & Growth Factors Reviews 16, 159-178 (2005). Furthermore, the disease may be a non-cancer disorder such as, without being limited to, benign skin tumors with FGFR3 activating mutations (Logic et al., Hum. Mol. Genet. 2005; Hafer et al., The Journal of Clin. Inv. 2006, 116:2201-2207), skeletal disorders resulting from mutations in FGFRs including achondroplasia, hypochondroplasia, severe achondroplasia with developmental delay and acanthosis nigricans (SADDAN), thanatophoric dysplasia (TD) (Webster et al., Trends Genetics 13 (5): 178-182 (1997); Tavormina et al., Am. J. Hum. Genet. 1999, 64: 722-731), muenke coronal craniosynostosis (Bellus et al., Nature Genetics 1996, 14: 174-176); Muenke et al., Am. J. Hum. Genet. 1997, 60: 555-564), crouzon syndrome with acanthosis nigricans (Meyers et al., Nature Genetics 1995, 11: 462-464), both familial and sporadic forms of Pfeiffer syndrome (Galvin et al., PNAS USA 1996, 93: 7894-7899; Schell et al., Hum. Mol. Gen. 1995, 4: 323-328); disorders related to alterations of phosphate homeostasis like hypophosphatemia or hyperphosphatemia, for example ADHR (autosomal dominant hypophosphatemic rickets), related to FGF23 missense mutations (ADHR Consortium, Nat. Genet. 2000 26(3):345-8), XLH (x-linked hypophosphatemic rickets), an x-linked dominant disorder related to inactivating mutations in the PHEX gene (White et al., Journal of Clinical Endocrinology & Metabolism 1996, 81:4075-4080; Quarles, Am. J. Physiol. Endocrinol. Metab. 2003, 285: E1-E9, 2003; doi:10.1152/ajpendo.00016.2003 0193-1849/03), TIO (tumor-induced osteomalacia), an acquired disorder of isolated phosphate wasting (Shimada et al., Proc. NatL Acad Sct. USA 2001 May 22; 98(11):6500-5), fibrous dysplasia of the bone (FD) (X. Yu et al., Cytokine & Growth Factor Reviews 2005, 16, 221-232 and X. Yu et al., Therapeutic Apheresis and Dialysis 2005, 2(4), 308-312), and tumoral calcinosis related to loss of FGF23 activity (Larsson et al., Endocrinology 2005 September; 146(9):3883-91).

The inhibition of FGFR activity has been found to represent a means for treating T cell mediated inflammatory or autoimmune diseases, as for example in treatment of T-cell mediated inflammatory or autoimmune diseases including but not limited to rheumatoid arthritis (RA), collagen II arthritis, multiple sclerosis (MS), systemic lupus erythematosus (SLE), psoriasis, juvenile onset diabetes, Sjogren's disease, thyroid disease, sarcoidosis, autoimmune uveitis, inflammatory bowel disease (Crohn's and ulcerative colitis), celiac disease and myasthenia gravis (see WO 2004/110487).

Disorders resulting from FGFR3 mutations are described also in WO 03/023004 and WO 02/102972.

In a further embodiment, one or more compounds selected from the group consisting of FGF23, P, P×tCa, OPN and PTH, preferably FGF23, can be used as safety markers in order to predict one or more secondary effects of a FGFR inhibitor, in particular ectopic mineralization. Preferably, FGF23 is used as safety marker to predict one or more secondary effects.

The term “secondary effect” as used herein refers to an undesired effect which may be harmful to the subject. Said effect is secondary to the main or therapeutic effect as described above. It may result from an unsuitable or incorrect dosage or procedure of FGFR modulators, but may also be connected with the mechanism of action of the FGFR inhibitors as in the case of ectopic mineralization.

Ectopic mineralization is an inappropriate biomineralization occurring in soft tissues, such as, without being limited to aorta, heart, lung, stomach, intestine, kidney, and skeletal muscle. In case of calcification, typically calcium phosphate salts, including hydroxyapatite are deposited, but also calcium oxalates and octacalcium phosphates are found (Giachelli C M, (1999), Am. J. Pathol., Vol. 154(3), p. 671-675). Ectopic mineralization is often associated with cell death. It leads to clinical symptoms when it occurs in cardiovascular tissues; in arteries, calcification is correlated with atherosclerotic plaque burden and increased risk of myocardial infarction as well as increased risk of dissection following angioplasty.

In a second aspect, the present invention provides a method for determining the modulation, preferably inhibition of kinase activity of FGFR, comprising the steps of

a) administering a FGFR inhibitor to a subject;
b) providing a sample of said subject;
c) determining the level of FGF23 of said sample; and
d) comparing the level of FGF23 of said sample with a reference level.

Said method is e.g. suitable for determining the therapeutic efficacy of a FGFR inhibitor and/or for determining one or more secondary effects of a FGFR inhibitor.

The subject of the methods disclosed herein is preferably a mammal, more preferably a rodent (such as a mouse or a rat), a dog, a pig, or a human.

The invention further provides a method for determining therapeutic efficacy of a FGFR inhibitor comprising steps a) to d) of the method disclosed herein, wherein the subject is a rat and the reference level is 745 pg/ml.

Moreover, the invention provides a method for determining one or more secondary effects of a FGFR inhibitor comprising steps a) to d) of the method disclosed herein wherein the subject is a rat and the reference level is 1371 pg/ml.

The “reference level” referred to in the methods of the instant invention may be established by determining the level of FGF23 in the subject before the onset of treatment with a FGFR inhibiting compound, i.e. by determining the baseline level of the subject. Thus, in an alternative embodiment, the method further comprises the step of measuring the baseline level of FGF23 in a subject. Another alternative consists in determining the level of FGF23 in a healthy control individual or group, or in a control individual or group with the same or similar proliferate disease which is treated with a non-therapeutic compound. Also, the reference level may well be derived from literature.

The sample of the subject is preferably derived from blood, e.g. plasma or serum, or urine. However, the method may also be practised on other body tissues or derivates thereof such as cell lysates. It is to be understood that the methods of the instant invention are practised ex vivo.

The present invention provides an ex vivo method for determining the modulation, preferably inhibition of kinase activity of FGFR comprising the steps of
a) determining FGF23 level in a sample of a patient before the onset of a FGFR inhibitor treatment (individual reference level);
b) determining FGF23 level in a sample of the same patient after receiving said FGFR inhibitor treatment.
   wherein the increased FGF23 level of step b) over the individual reference level indicates the modulation, preferably inhibition, of the kinase activity of FGFR occurred.

In one preferred embodiment, the patient is a cancer patient. In one preferred embodiment, the cancer of such patient is caused or related to deregulated FGFR signalling. More preferably the cancer is a solid tumor, preferably including but not limited to bladder cancer, melanoma and kidney cancer.

Although the degree of FGF23 increase varies depending on the nature of each individual FGFR inhibitor, the dosage and the treatment regimen, the use of FGF23 as a biomarker provides a reliable, convenient and non-invasive way for monitoring patient's response towards FGFR inhibitor treatment. Furthermore doctor may according to the increased value of FGF23 make better prognosis, adjust the dose, switch to other treatment or closely monitoring and avoiding secondary effects due to the treatment.

Preferably the FGF23 level of step b) is increased at least 1.2 fold compared to the individual reference level, further preferably at least 1.4 fold, at least 1.5 fold, at least 1.7 fold, at least 2 fold, at least 2.5 fold. For potent FGFR inhibitors, such as compound A, the FGF23 level may increase at least 2.5 fold, at least 3 fold, 4 fold or even higher.

The increase of FGF23 level after FGFR inhibitor treatment normally is observed after the first standard dosage of the particular FGFR inhibitor. Information regarding standard dosage of a particular FGFR inhibitor can be found normally on the label of the drug containing the particular FGFR inhibitor as API. Normally the FGF23 level is measured once the FGFR inhibitor concentration reaches its steady state. Our preliminary observation with melanoma patients and metastatic renal cell carcinoma patients treated with 400 mg or 500 mg TKI258 indicated that the peak of FGF23 is around day 15 in the first cycle of treatment. Thus in one preferred embodiment, the method of the present invention comprises determining FGF23 level in a sample of the same patient after receiving said FGFR inhibitor treatment for at least 5 days, preferably for at least 5 days but not longer than 30 days, preferably for at least 10 days but not longer than 25 days, for at least 10 days but not longer than 20 days.

In the case of patients treated with 400 mg daily with TK1258, the levels of FGF23 could increase upto 1.96 fold and 2.1 fold.

In one preferred embodiment, the FGFR inhibitor is compound A or any pharmaceutically acceptable salt thereof. In one preferred embodiment, the FGFR inhibitor is TKI258 or any pharmaceutically acceptable salt thereof.

In one aspect, the present invention provides a use of an FGFR inhibitor for the manufacture of a medicament for the treatment of a proliferative disease, wherein preferably said proliferative disease is cancer, more preferably cancer with deregulated FGFR signalling, in a patient, wherein said patient has increased level of FGF23 after taking said FGFR receptor inhibitor. Alternatively the present invention provides a method of treating a proliferative disease, wherein preferably said proliferative disease is cancer, more preferably cancer with deregulated FGFR signalling, in a patient, comprising the step of administering an FGFR inhibitor to said patient, wherein said patient has increased level of FGF23 after taking said FGFR receptor inhibitor. The increase of FGF23 level after FGFR inhibitor treatment normally is observed after the first standard dosage of the particular FGFR inhibitor. Normally the FGF23 level is measured once the FGFR inhibitor concentration reaches its steady state. Thus the use of FGF23 as biomarker allows stratification of patients, particularly cancer patients with deregulated FGFR signalling, depending their responses to a FGFR inhibitor.

The present application provides a method for screening patients to determine whether a patient will benefit from a FGFR inhibitor treatment, said method comprises the steps of

(a) giving a patient a FGFR inhibitor treatment for a period of time;
(b) measuring the FGF23 level in the sample of said patient after said treatment;
(c) comparing the FGF23 value obtained from step (b) to the individual reference level (FGF23 level in said patient before the onset of said FGFR inhibitor treatment) and deciding whether said patient should continue said FGFR inhibitor treatment or not.

The term “period of time” as used herein refers to a relative short period of time, normally not longer than 30 days, more likely not longer than 15 days, possibly not longer than one week.

During this “trial” period of time, patient is given said FGFR inhibitor treatment according to standard regimen or even to elevated dosage or more frequently administration or both.

The patient has normally a condition that could be caused or related to deregulated FGFR signalling, in most cases the patient has cancer that could be caused or related to deregulated FGFR signalling.

The increase of FGF23 compared to the individual reference level is normally at least 1.2 fold, preferably at least 1.3 fold or at least 1.5 fold. This is typically and preferably the case when the FGFR inhibitor is TKI258.

For a potent FGFR inhibitor a more increase of FGF23 could be expected. In the case of compound A, the increase is at least 1.3 fold, preferably at least 1.5 fold, more preferably at least 2 fold, more preferably at least 3 fold.

FGFR inhibitor is preferably selected from the group consisting of PD176067, PD173074, compound A (3-(2,3-Dichloro-3,5-dimethoxy-phenyl)-1-{6-[4-(4-ethyl-perpazin-1-yl)-phenylamino]-pyrimidin-4-yl}-1-methyl urea), TKI258 and compound B (a derivative of [4,5′]bipyrimidinyl-6,4′-diamine).

In one preferred embodiment, the FGFR inhibitor is compound A or any pharmaceutically acceptable salt thereof. In one preferred embodiment, the FGFR inhibitor is TKI258 or any pharmaceutically acceptable salt thereof.

For purposes of detection, the sample may be further treated, e.g. proteins may be isolated using techniques that are well-known to those of skill in the art.

Typically, the level of FGF23 is determined by measuring the presence of the polypeptide FGF23 in said sample of a subject with a suitable agent for detection. A preferred agent for detecting a polypeptide of the invention is an antibody capable of binding to a polypeptide corresponding to a marker of the invention, preferably an antibody with a detectable label. Antibodies can be polyclonal, or more preferably, monoclonal. An intact antibody, or a fragment thereof; e.g., Fab or F(ab)₂ can be used.

In another embodiment, the expression of the FGF23 coding sequence may be detected in the sample, e.g. by determining the level of the corresponding RNA. A suitable detection agent is a probe, a short nucleic acid sequence complementary to the target nucleic acid sequence.

In a preferred embodiment of the invention, the FGF23 polypeptide is detected.

The detection agent may be directly or indirectly detectable and is preferably labeled. The term “labeled”, with regard to the probe or antibody, is intended to encompass direct-labeling of the probe or antibody by coupling, i.e., physically linking, a detectable substance to the probe or antibody, as well as indirect-labeling of the probe or antibody by reactivity with another reagent that is directly-labeled. Examples of indirect labeling include detection of a primary antibody using a fluorescently-labeled secondary antibody and end-labeling of a DNA probe with biotin such that it can be detected with fluorescently-labeled streptavidin.

The label may be one as conventional, e.g. biotin or an enzyme such as alkaline phosphatase (AP), horse radish peroxidase (HRP) or peroxidase (POD) or a fluorescent molecule, e.g. a fluorescent dye, such as e.g. fluorescein isothiocyanate.

In a preferred embodiment of the invention, the detection means comprise an antibody, including antibody derivatives or fragments thereof; e.g. an antibody which recognizes FGF23, e.g. a label bearing FGF23 recognizing antibody. In another aspect, the level of FGF23 is determined in using a FGF23 specific antibody.

The detection agent, e.g. the label bearing antibody, may be detected according to methods as conventional, e.g. via fluorescence measurement or enzyme detection methods, including those as conventional in the field of assays, e.g. immunoassays, such as enzyme linked immunoassays (ELISAs); fluorescence based assays, such as dissociation enhanced lanthanide fluoroimmunoassay (DELFIA) or radiometric assays such as radioimmunoasay (RIA). Further suitable examples include, but are not limited to, EIA and Western blot analysis. A skilled artisan can readily adapt known protein/antibody detection methods for use in determining the level of FGF23.

It is to be understood that the methods disclosed herein can be similarly performed with a compound selected from the group consisting of P, P×tCa, OPN and PTH.

In a preferred embodiment, two or more compounds selected from the group consisting of FGF23, P, P×tCa, OPN and PTH are used in the methods disclosed herein, most preferably, FGF23 in combination with one or more compounds selected from the group consisting of P, P×tCa, OPN and PTH. By using multiple biomarkers, the accuracy of determining the therapeutic efficacy and/or one or more secondary effects of a FGFR inhibitor is enhanced. When one or more compounds selected from the group consisting of FGF23, P, P×tCa, OPN, PTH are used as a safety biomarker, the above described method for determining one or more secondary effects of a FGFR inhibitor may further comprise the steps of

e) correlating the level of one or more compounds selected from the group consisting of FGF23, P, P×tCa, OPN, PTH with one or more secondary effects; and
f) determining the level of said compound(s) above which the secondary effect will occur, relatively to the treatment employed.

Preferably, the level of FGF23, P, P×tCa, OPN is increased when compared to the reference level.

Preferably, the level of PTH is decreased when compared to the reference level.

In another aspect, the invention provides a method for determining the responsiveness of a subject having a FGFR related disorder to a therapeutic treatment with a FGFR inhibitor, comprising the step of determining the level of one or more compounds selected from the group consisting of FGF23, P, P×tCa, OPN, PTH, preferably of FGF23, in the plasma or in the serum of the subject.

As used herein, “therapeutic treatment” refers to the treatment, prevention or delay of progression of a FGFR related disorder, preferably of a proliferative disease, more preferably of a cancer.

In still another aspect, the invention provides a diagnostic kit comprising elements a) to d) as outlined below. In particular, it relates to a kit for determining the efficacy of a FGFR inhibitor and/or the secondary effects of FGFR inhibitors, preferably in a sample of a subject, comprising

a) a molecule which recognizes one or more compounds selected from the group consisting of FGF23, P, P×tCa, OPN and PTH or a part thereof, optionally in a labelled form;
b) optionally instructions for use;
c) optionally detection means; and
d) optionally a solid phase.

Further, the use of said kit for determining the efficacy of a FGFR inhibitor and/or the secondary effects of FGFR inhibitors, preferably in a sample of a subject, is provided.

In one preferred embodiment, the present invention provides a diagnostic kit comprising

a) a molecule which recognizes FGF23 or a part thereof, optionally in a labelled form;
b) at least one reagent capable of detecting a second biomarker selected from the group consisting of inorganic phosphorus (P), the product of phosphorus and total calcium (P×tCa), osteopontin (OPN) and parathyroid hormone (PTH);
c) optionally instructions for use;
d) optionally detection means; and
e) optionally a solid phase.

Furthermore the present invention provides use of the kit as outlined above for determining the efficacy of a FGFR inhibitor and/or the secondary effects of FGFR inhibitors in a sample of a subject.

In one preferred embodiment, the kit comprises at least one reagent capable of detecting a second biomarker being inorganic phosphorus (P).

The following examples are presented in order to more fully illustrate the preferred embodiments of the invention. These examples should in no way be construed as limiting the scope of the invention, as defined by the appended claims.

### Example 1

### Dose Dependent Inhibition of Tumor Homografts by COMPOUND A; FGF23 as Biomarker to Monitor the Inhibition of Fibroblast Growth Factor Receptor Kinase Activity

### 1.1 Methods

Animals. Experiments were performed in female HsdNpa: Athymic Nude-nu mice obtained from Laboratory Animal Services, Novartis Pharma AG, Basel, Switzerland. The animals were kept under OHC conditions in Makrolon type II cages (maximum of 10 animals/cage) with 12 hour dark, 12 hour light conditions (lights on: 6 AM, lights off: 6 PM). The animals were fed food and water ad libitum. Experiments were conducted under license number 1762 and license number 1763 approved by the Basel Cantonal Veterinary Office. All invasive procedures were performed under Forene anesthesia.

Establishment of NIH3T3/FGFR3^{S249C} tumor homograft model in nude mice. The NIH3T3/FGFR3^{S249C} model has been validated and characterized as a subcutaneous murine tumor model for the in vivo profiling of FGFR inhibitors. The parental NIH3T3 cell line was originally derived by immortalization of mouse embryonic fibroblasts. NIH3T3/FGFR3^{S249C} cells were generated by infection of parental NIH3T3 fibroblasts with a retroviral vector expressing FGFR3 with the activating mutation S249C. Pools of G418 resistant NIH3T3^{S249C} cells were established and characterized for FGFR3 expression and tyrosine phosphorylation. To generate homografts, 5×10⁵ NIH3T3/FGFR3^{S249C} cells resuspended in PBS were injected subcutaneously in nude mice (0.2 ml/mouse).

Establishment of RT112/lucl tumor xenograft model in nude mice. The parental RT-112 human urinary bladder transitional cell carcinoma cell line, which expressed high levels of wild type FGFR3, was initially derived from a female patient with untreated primary urinary bladder carcinoma (histological grade G2, stage not recorded) in 1973 (Marshall et al., 1977, Masters et al., 1986). The original stock vial of RT112 cells used in this study was obtained from DSMZ ACC #418.

The cells were cultured in MEM medium supplemented with 10% Fetal Calf Serum, 1% sodium pyruvate and 1% L-glutamine. Cell culture reagents were purchased from BioConcept (Allschwil, Switzerland).

The parental RT112 cell line was infected with the retroviral expression vector pLNCX2/lucl and pools of G418 resistant cells were established and characterized for luciferase expression. The CMV driven expression of luciferase allows the detection of tumors using Xenogen IVIS™ cameras after injection of D-luciferin.

RT112/lucl xenograft tumors were established by subcutaneous injection of 5×10⁶ cells in 100 μl HBSS (Sigma #H8264) containing 50% Matrigel (BD #356234) into the right flank.

Evaluation of anti-tumor activity. For the NIH3T3/FGFR3^{S249C} model, treatment was initiated when the average tumor volume reached approximately 100 mm³. Tumor growth and body weights were monitored at regular intervals. The tumor sizes were measured manually with calipers. Tumor volume was estimated using the formula: (W×L×H×π/6), where width (W), length (L) and height (H) are the three largest diameters.

For the RT112/lucl model, treatments were initiated when the mean tumor volumes were approx. 180 mm³ and mice were treated daily for 14 days. Body weights and tumor volumes were recorded twice a week. Tumor volumes were measured with calipers and determined according to the formula length×diameter²×π/6.

Statistical analysis. When applicable, results are presented as mean±SEM. Tumor and body weight data were analyzed by ANOVA with post hoc Dunnett's test for comparison of treatment versus control group. The post hoc Tukey test was used for intra-group comparison. The level of significance of body weight change within a group between the start and the end of the experiment was determined using a paired t-test. Statistical analysis was performed using GraphPad prism 4.02 (GraphPad Software).

As a measure of anti-tumor efficacy, the % T/C value is calculated at a certain number of days after treatment start according to: (mean change of tumor volume treated animals/mean change tumor volume control animals)×100. When applicable, % regressions are calculated according to the formula (mean change tumor volume/mean initial tumor volume)×100.

Compound formulation and animal treatment. COMPOUND A was formulated as a suspension in PEG300/DSW (2:1 v/v, D5W=5% glucose in water) and applied daily by gavage. Vehicle consisted of PEG300/DSW. The application volumes were 10 ml/kg.

Tissue processing for ex vive analysis. At the end of the experiments, 2 hours after the last compound administrations, tumor samples and blood were collected.

Tumor samples were dissected and snap frozen in liquid N₂. The tumor material was pulverized using a swing mill (RETSCH MM200). The grinding jars and balls were chilled on dry ice for half an hour prior to adding frozen tumor samples. The swing mill was operated for 20 seconds at 100% intensity. The tumor powder was transferred to 14 mL polypropylene (all steps on dry ice) and stored at −80° C. until use.

Aliquots of 50 mg tumor powder were weighed, placed on ice and immediately resuspended at a ratio of 1:10 (w/v) in ice-chilled lysis buffer (50 mM Tris pH 7.5, 150 mM NaCl, 1 mM EGTA, 5 mM EDTA, 1% Triton, 2 mM NaVanadate, 1 mM PMSF and protease inhibitors cocktail Roche #11873580001). Lysis was allowed to proceed for 30 min on ice, lysates were clarified by centrifugation at 12000×g for 15 min and protein concentration was determined using DC protein assay reagents (Bio Rad #500-0116) and a BSA standard. Blood was collected from the vena cava with a 23 gauge needle into a 1 ml syringe containing 70 μl of a 1000 IU/ml heparin solution. Blood was then stored on ice for 30 min until centrifugation (10,000 g, 5 min) and then the plasma was collected.

Immunoprecipitation and Western blot analysis. Equal amounts of protein lysates were pre-cleared with protein A-sepharose followed by incubation with 1 μg of α-FGFR3 antibody (rabbit polyclonal, Sigma #F3922) for 2 h on ice. Immunocomplexes were collected with protein A-sepharose and washed 3× lysis buffer. Bound proteins were released by boiling in sample buffer (20% SDS, 20% glycerol, 160 mM Tris pH 6.8, 4% β-mercaptoethanol, 0.04% bromo-phenol blue).

Samples were subjected to SDS-PAGE and proteins blotted onto PVDF membranes. Filters were blocked with 20% horse serum, 0.02% Tween 20 in PBS/O for 1 h and the anti-pTyrosine antibody 4G10 (Upstate) was added at 1:1000 dilution for 2 h at RT. Proteins were visualized with peroxidase-coupled anti-mouse antibody (Amersham #NA931V) using the SuperSignal® West Dura Extended Duration Substrate detection system (Pierce #34075). Further, membranes were stripped in 62.5 mM Tris-HCl pH6.8; 2% SDS; 1/125 β-mercaptoethanol for 30 min at 60° C., reprobed with α-FGFR3 antibody (rabbit polyclonal, Sigma #F3922) followed by peroxidase-coupled anti-rabbit antibody (Amersham #NA934V). Proteins were visualized as described above.

FGF23 ELISA assay. To monitor FGF23 levels in plasma or serum samples, the FGF23 ELISA assay from KAINOS Laboratories, Inc., Japan was used (catalogue #CY-4000). Briefly, two specific murine monoclonal antibodies that bind to full-length FGF-23 are used: the first antibody is immobilized onto the microtiter plate well for capture and the second antibody is cojugated to HRP (horseradish peroxidase) for detection. In a first reaction, plasma or serum samples are added onto microtiter wells coated with the anti-FGF23 antibody to allow binding. Wells are washed to remove unbound FGF23 and other components. In a second reaction, the immobilized FGF23 is incubated with HRP labeled antibody to form a “sandwich” complex.

This ELISA assay has been validated for the monitoring of FGF23 in serum and plasma of mouse, rat and dog.

### 1.2 Results and Discussion

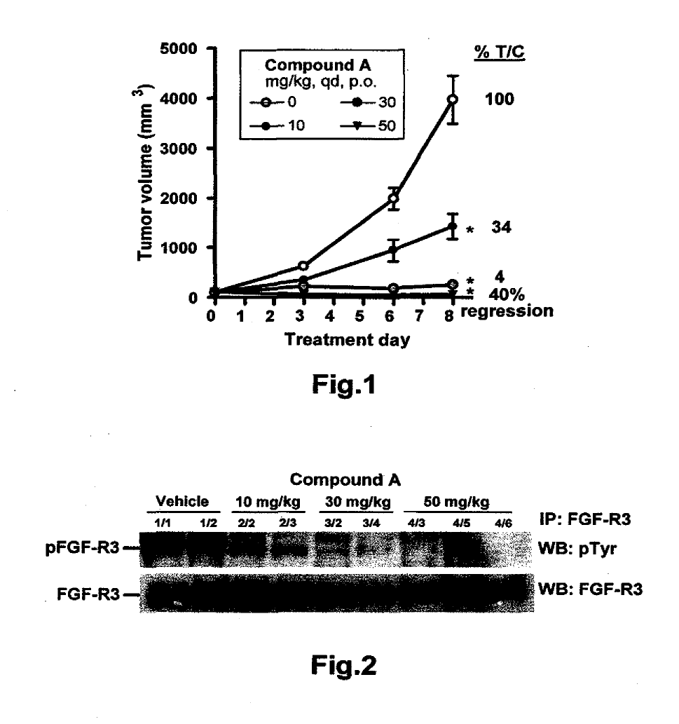

Activity of COMPOUND A in the NIH3T3/FGFR3^{S249C} model. The anti-tumor effect of COMPOUND A was evaluated in the subcutaneous NIH3T3/FGFR3^{S249C} model. Dose levels of 10, 30 and 50 mg/kg were tested. Treatment was initiated when the estimated average tumor size reached 100 mm³ (day 0) and the animals were treated for 8 days. Tumor sizes and body weights were evaluated on treatment day 8 by one-way ANOVA. Statistically significant anti-tumor effect was observed at all dose levels when compared to vehicle treated animals (ANOVA post hoc Dunnett's), with T/C values of 34 and 4% at 10 and 30 mg/kg, respectively and 40% tumor regression at 50 mg/kg (Table 1, FIG. 1). The two highest dose levels gained statistically significant less body weight during the treatment period. However, the additional body weight gain observed in the vehicle treated and the group treated with 10 mg/kg is, at least in part, accounted for by the tumor mass.

TABLE 1

| | | Tumor response | | | Host response | |
|---|---|---|---|---|---|---|
| | Dose, | | | Δ tumor | Δ body | Δ body |
| | route, | T/C | Regr. | volume (mean | weight (mean | weight (% ± |
| Compound | schedule | (%) | (%) | mm³ ± SEM) | g ± SEM) | SEM) |
| Vehicle | 10 ml/kg, | 100 | NA | 3853 ± 473 | 4.1 ± 0.5 | 16.9 ± 2 |
| | p.o., qd | | | | | |
| COMPOUND A | 10 mg/kg, | 34 | NA | 1320 ± 245* | 4.2 ± 0.7 | 18.0 ± 3.2 |
| | p.o., qd | | | | | |
| COMPOUND A | 30 mg/kg, | 4 | NA | 156 ± 56* | 1.6 ± 0.7 | 7.0 ± 3.1* |
| | p.o., qd | | | | | |
| COMPOUND A | 50 mg/kg, | NA | 40 | −43 ± 28* | 1.1 ± 0.5 | 4.6 ± 2.2* |
| | p.o., qd | | | | | |

Pharmacodynamics of FGFR3 tyrosine phosphorylation upon treatment with COMPOUND A. NIH3T3/FGFR3^{S249C} tumors from animals treated with 10, 30 or 50 mg/kg qd, or vehicle were dissected at 2 h post last dosing, which is within the tmax interval established in previous pharmacokinetic studies. The ex vivo analysis of NIH3T3/FGFR3^{S249C} implanted tumors demonstrated a dose dependent inhibition of FGFR3 Tyr-phosphorylation while total receptor levels remained constant (FIG. 2). This pharmacodynamic effect correlated with the anti-tumor effect (FIG. 1).

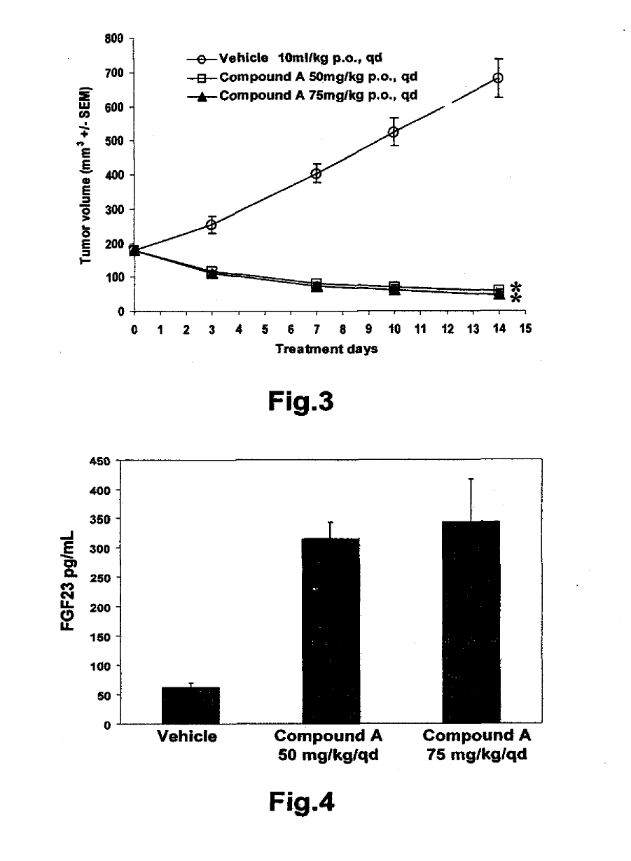

Activity of COMPOUND A in the RT112/lucl model. The anti-tumor activity of COMPOUND A was assessed at two different dose levels, 50 and 75 mg/kg per day administered orally to nude mice. The two doses produced a statistically significant tumor regression (p<0.01, ANOVA post hoc Dunnett's). The regression values were 67 and 74% for COMPOUND A at 50 and 75 mg/kg respectively (Table 2, FIG. 3). Treatments were well tolerated, as shown by statistically significant increase in body weight in the vehicle and COMPOUND A at 50 mg/kg/day groups over the course of the experiment. The group treated with 75 mg/kg COMPOUND A showed a slight body weight loss, although not statistically significant. The increases in body weights were found to be significantly different in the group treated with 75 mg/kg COMPOUND A when compared to vehicle controls (p<0.01, ANOVA, post hoc Dunnett's). In addition, the group treated with 75 mg/kg COMPOUND A showed a statistically significant difference in body weight change when compared to all other groups (p<0.05, ANOVA, post hoc Tukey).

TABLE 2

| | | Tumor response | | | Host response | |
|---|---|---|---|---|---|---|
| | Dose, | | | Δ tumor volume | Δ body | Δ body |
| | route, | T/C | Regr. | (mean mm³ ± | weight (mean | weight (% ± |
| Compound | schedule | (%) | (%) | SEM) | g ± SEM) | SEM) |
| Vehicle | 10 ml/kg, | 100 | NA | 500 ± 54 | 3.3 ± 0.6* | 13.5 ± 2.7 |
| | p.o., qd | | | | | |
| COMPOUND A | 50 mg/kg, | NA | 67 | −120 ± 12* | 2.6 ± 1.0* | 10.8 ± 4.1 |
| | p.o., qd | | | | | |
| COMPOUND A | 75 mg/kg, | NA | 74 | −133 ± 13* | −1.1 ± 1.1 | −4.0 ± 4.5 |
| | p.o., qd | | | | | |

FGF23 levels in plasma samples of nude mice. As part of the study described in section 1.1, FGF23 levels were determined in plasma samples from mice treated with 50 or 75 mg/kg/qd COMPOUND A or vehicle, two hours post-last dosing. Mice that were treated with COMPOUND A showed increased plasma levels of FGF23 as compared to the vehicle-treated group (FIG. 4), which correlated with the anti-tumor efficacy effect observed with both doses of the compound (FIG. 3).

Conclusion. The experimental data presented demonstrates that doses of COMPOUND A that inhibit FGFR3 in vivo and produce statistically significant anti-tumor effects in two murine tumor models, also lead to increased levels of plasma FGF23 in a dose dependent manner.

### Example 2

### Rat Mechanistic Study

### 2.1 Methods

Animals. Experiments were performed in male Crl:WI (Han) rats (14-17 week old at start of dosing) obtained from Charles River Laboratories Germany GmbH, Research Models and Services, Sulzfeld, Germany. The animals were kept under optimal hygene conditions (OHC) in Makrolon type IV cages with 12 hour dark, 12 hour light conditions. Pellets standard diet and water was provided ad libitum. This study was performed in conformity with the Swiss Animal Welfare Law and specifically under the Animal License No. 5075 by ‘Kantonales Veterinäramt Baselland’ (Cantonal Veterinary Office, Baselland).

Compound formulation and animal treatment. COMPOUND A was formulated as a solution in acetic acid-acetate buffer (pH 4.6)/PEG300 (2:1 v/v) and applied daily by gavage. Vehicle consisted of acetic acid-acetate buffer (pH 4.6)/PEG300 (2:1 v/v). The application volumes were 5 ml/kg.

Study design. COMPOUND A was orally administered to groups of 10 male rats at doses of 10 mg/kg for 1, 3, 7 and 15 days, or 20 mg/kg for 1, 3 and 6 days, once daily. Animals treated at 20 mg/kg had to be prematurely terminated after the 6^{th} administration due to severe body weight loss. Control animals received the vehicle for 1, 3, 7, and 15 days. Additional groups (10 males each), receiving either COMPOUND A (doses: 10 mg/kg for 3, 7, and 15 days; 20 mg/kg for 1 and 3 days) or the vehicle, were introduced to further investigate treatment related effects and monitor variations in the selected clinical chemistry parameters after 4, 7, or 14 days of recovery.

### 2.2 Results and Discussion

Hstopathology findings related to FGFR inhibition. Growth plate thickening was detected after three days of treatment in animals dosed with 10 and 20 mg/kg/day. This is a consequence of inhibiting FGFR3, most likely in chondrocytes. Indeed, growth plate thickening, due to increased size of the hypertrophic zone, had previously been shown to also occur in mice homozygous for a targeted disruption of FGFR3, i.e. lacking FGFR3 expression (Colvin et al., Nature Genetics 1996, 12: 390-397). This observation demonstrates that FGFR3 plays a role in regulating growth plate enlargement. Thus, the findings related to the growth plate are considered a pharmacological read out for the FGFR inhibitors and are an indication of efficacy, i.e. inhibition of FGFR3, of a FGFR inhibitor. Signs of bone remodeling events were noted in animals treated with 10 mg/kg/day after 15 days of treatment and 4 days recovery period and 20 mg/kg/day after 3 days of treatment and 4 days recovery period (delayed effects), and in animals treated for 6 days with 20 mg/kg/day. Soft tissue/vascular mineralization was detected in animals treated with 20 mg/kg/day for 3 days after 4 recovery days and after 6 days of treatment at the 20 mg/kg/qd dose of COMPOUND A. Such finding was not observed in the groups administered with 10 mg/kg/qd of COMPOUND A.

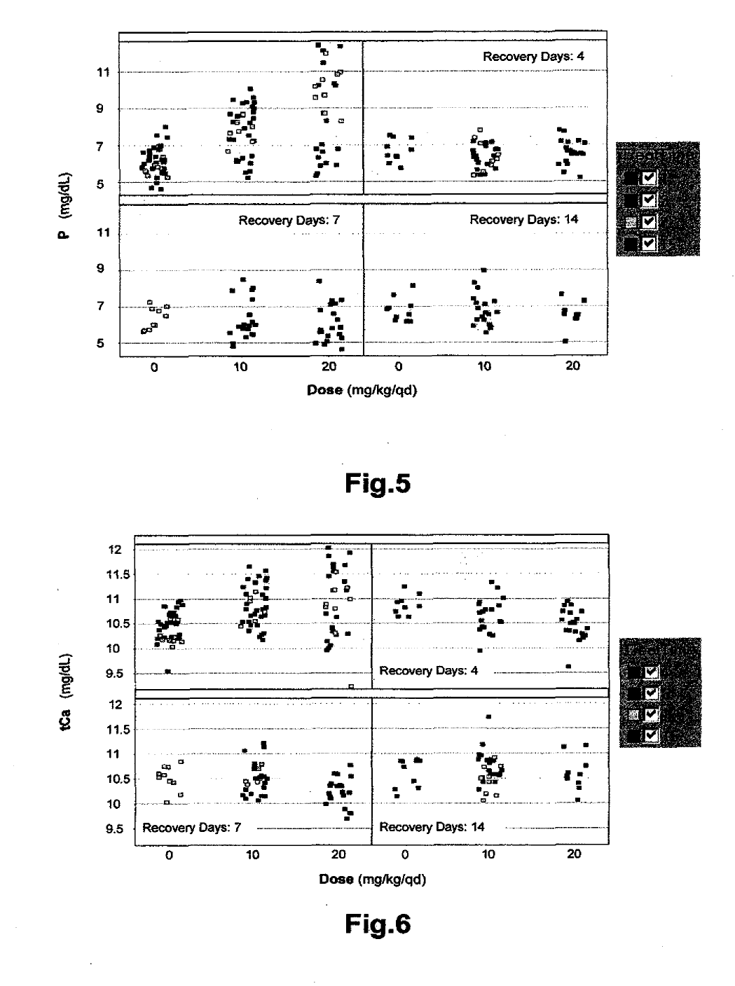

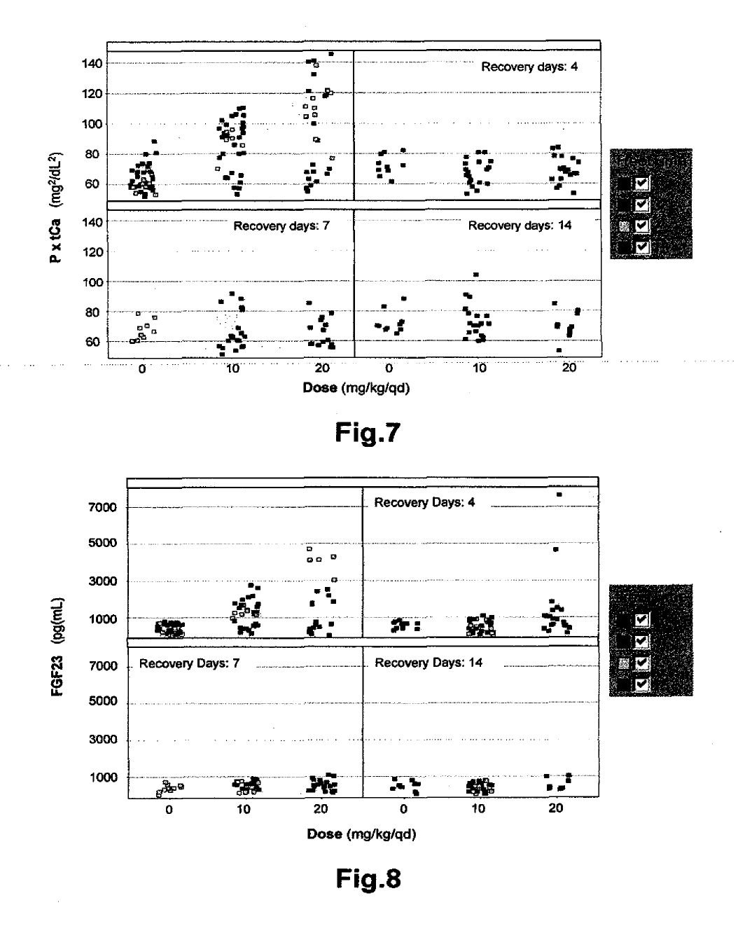

Clinical chemistry parameters. Inorganic phosphorus (P), the product of inorganic phosphorus and total calcium (P×tCa), parathyroid hormon (PTH), osteopontin (OPN) and FGF23 were measured with the aim of assessing their utility as markers to predict and monitor the onset of pharmacological (growth plate thickening) and pathological (bone remodeling and ectopic mineralization) events. The variations in serum of the levels of P, tCa, their product and FGF23 are illustrated as scatter plots in FIGS. 5, 6, 7 and 8, respectively. Each plot (grey scale square representing single animal) is reported as a function of the peripheral concentration of the marker (Y axis) and of the COMPOUND A dose (X axis). Different grey shades are associated to specific treatment periods. Spotfire 8.2 was used for the data visualization.

Method for biomarkers validation A quantitative assessment of performance of the selected markers measured in the rat exploratory study was conducted by Receiver Operating Characteristics (ROC) analysis, a method commonly used to evaluate medical tests which allows for the determination of the diagnostic power of a given assay by measuring the area under the ROC curve (AUC). Swets J A, Science. 240:1285-93 (1988); Swets J A et al., Scientific American. 283:82-7 (2000).

Assessment of selected biomarkers performances. The ranking of the markers performance (AUC), obtained by application of ROC analysis to the data obtained from the treatment phase, is reported in Table 3.

TABLE 3

| Pharmacology | | | | Pathology | | | |
|---|---|---|---|---|---|---|---|
| Marker | AUC | SE | p. value | Marker | AUC | SE | p. value |
| FGF23 | 0.92 | 0.03 | 0.0E+00 | FGF23 | 1.00 | 0.00 | 0.0E+00 |
| P × tCa | 0.90 | 0.03 | 0.0E+00 | OPN | 1.00 | 0.00 | 0.0E+00 |
| PTH | 0.90 | 0.03 | 0.0E+00 | P | 0.90 | 0.03 | 0.0E+00 |
| P | 0.89 | 0.03 | 0.0E+00 | P × tCa | 0.87 | 0.04 | 0.0E+00 |
| OPN | 0.84 | 0.07 | 8.4E−07 | PTH | 0.75 | 0.07 | 3.1E−04 |

SE = standard error of the AUC.
p. value = probability of obtaining the corresponding AUC value by chance.

ROC analysis was used to conduct an additional evaluation of the performance of FGF23, taking into account the delayed pathological effects. Such analysis allowed for the determination of pharmacology and safety thresholds for this marker (Table 4). The pharmacology threshold value is 745 pg/mL, representing the FGF23 level above which growth plate thickening can be observed during the treatments considered in this analysis. The safety threshold value is 1371 pg/mL, representing the highest FGF23 level allowed during the treatments considered in this analysis which ensures absence of delayed pathological effects (bone remodeling and ectopic mineralization).

TABLE 4

| | FGF23 | |
|---|---|---|
| | | Threshold |
| Assessment | AUC | (pg/mL) |
| Pharmacology | 1.00 | 745 |
| Pathology | 0.99 | 1371 |

Conclusion. Among the clinical parameters measured in the context of this study in the rat, several are found to be suitable pharmnacodynamic markers. These markers exhibit good to very high levels of performances as demonstrated by the corresponding AUC values reported in Table 3. Further as shown in Table 4, this study demonstrates that FGF23 is a predictive biomarker to monitor the onset of growth plate thickening (therapeutic efficacy/pharmacology) and to prevent the onset of bone remodeling and ectopic mineralization (safety/pathology). Pharmacology and safety thresholds have been established for FGF23 in the context of this specific study and of the treatments considered in the analysis.

### Example 3

### FGF23 Induction by COMPOUND A in Dogs

### 3.1 Methods

Animals. Experiments were performed in dogs:
Animal species and strain: Dog, Beagle.
Number of animals in study: 8
Age: 13 to 18 months (at start of dosing).
Body weight range: 7 to 11 kg (at start of dosing).
Suppliers veterinary treatments: Antiparasitic therapy and vaccination against canine distemper, infectious canine hepatitis, parainfluenza, leptospirosis, parvovirus, adenovirus, rabies.

Compound formulation and animal treatment. COMPOUND A was formulated as a suspension in 0.5% HPMC603 and applied once daily by oral gavage. Vehicle consisted of 0.5% HPMC603. The application volumes were 2 ml/kg.

Study design: dogs were treated with vehicle or compound A as indicated:

TABLE 1

| Group | Dosage | Animals | Male | Female | Dosage volume |
|---|---|---|---|---|---|
| no. | (mg/kg/day)* | per sex | no. | no. | (mL/kg/day) |
| 1 | 0* | 1 | 451 | 452 | 2 |
| 2 | 3/100** | 1 | 453 | 454 | 2 |
| 3 | 30* | 1 | 455 | 456 | 2 |
| 4 | 300*** | 1 | 457 | 458 | 2 |

*Group 1 and group 3 were treated for 15 consecutive days.
**Group 2 was treated for 8 days with 3 mg/kg/day. From day 9 to day 18 the dose was increased to 100 mg/kg/day; from day 19 to day 21 the animals were on drug holiday, and dosing was resumed on day 22 and day 23 (100 mg/kg: 10 days ON, 3 days OFF, 2 days on).
***Group 3 received 300 mg/kg/day for 8 consecutive days.

Blood sampling for ex vivo analysis. At the end of the dosing period, 1 hour after the last compound administrations, whole blood was collected into EDTA-coated tubes taken from the vena jugularis or the vena cephalica antebrachii and kept on ice water until further processing. The specimen was centrifuged and plasma was transferred into an Eppendorf tube and set on dry ice.

FGF23 ELISA assay. To monitor FGF23 levels in plasma samples, the FGF23 ELISA assay from KAINOS Laboratories, Inc., Japan was used (catalogue #CY-4000). Briefly, two specific murine monoclonal antibodies that bind to full-length FGF-23 are used: the first antibody is immobilized onto the microtiter plate well for capture and the second antibody is conjugated to HRP (horseradish peroxidase) for detection. In a first reaction, plasma samples are added onto microtiter wells coated with the anti-FGF23 antibody to allow binding. Wells are washed to remove unbound FGF23 and other components. In a second reaction, the immobilized FGF23 is incubated with HRP labeled antibody to form a “sandwich” complex.

### 3.2 Results and Discussion

FGF23 levels in plasma samples of dogs. Dogs that were treated with COMPOUND A showed increased plasma levels of FGF23 as compared to the vehicle-treated group (Table 2), which were in general dose-dependent. The lower than dose-proportional increase for group 2 could be explained by an adaptation mechanism during the dosing period at 3 mg/kg/day. Alternatively, the three days OFF after 10 days ON might result in a decrease in FGF23.

TABLE 2

| Group | | animal | FGF-23 cont. |
|---|---|---|---|
| no. | mg/kg/day | no. | (pg/mL) |
| 1 | baseline | # 451 | 173.9 |
| | | # 452 | 205.8 |
| 2 | 3 → 100 | # 453 | 262.5 |
| | | # 454 | 211.3 |
| 3 | 30 | # 455 | 534.3 |
| | | # 456 | 322.7 |
| 4 | 300 | # 457 | 824.6 |
| | | # 458 | 614.8 |

Conclusion. The experimental data presented demonstrates that COMPOUND A leads to increased levels of plasma FGF23 in dogs.

### Example 4

### FGF23 Measurements in Plasma Samples from Melanoma Cancer Patients Treated with TKI258

### 4.1 Methods

Compound: TKI258 is a multi-kinase inhibitor that inhibits among others, FGFR1, FGFR2 and FGFR3 with IC50 values in cellular assays of 166, 78 and 55 nM, respectively.

Patients and treatment: metastatic melanoma patients were treated daily with TKI258 administered orally at the indicated doses. Blood sampling was performed at the indicated day and cycle. FGF23 levels were measured in plasma. The values given for C1D1 are the baseline values.

FGF23 ELSA assay. To monitor FGF23 plasma samples in patients, the FGF23 ELISA assay from KAINOS Laboratories, Inc., Japan was used (catalogue #CY-4000) as described in Example 3.

### 4.2 Results and Discussion

FGF23 data from three different patients is shown in Table 3

TABLE 3

| | Dose | Treatment cycle (C)/ | FGF23 | Fold |
|---|---|---|---|---|
| Patient | [mg] | Treatment day (D) | [pg/mL] | Induction |
| A | 200 | C1D1 | 36.4 | 1.00 |
| | | C1D15 | 39.8 | 1.09 |
| | | C1D26 | 24.4 | 0.67 |
| | | C3D26 | 31.8 | 0.87 |
| | | C6D26 | 21.1 | 0.58 |
| | | C9D26 | 39.6 | 1.09 |
| B | 400 | C1D1 | 72.3 | 1.00 |
| | | C1D15 | 94.1 | 1.30 |
| | | C3D26 | 88.5 | 1.22 |
| | | C4D26 | 141.5 | 1.96 |
| C | 400 | C1D1 | 41.1 | 1.00 |
| | | C1D15 | 86.5 | 2.10 |

Patient A treated at 200 mg of TKI258 showed similar levels of FGF23 throughout the treatment. In patients B and C treated with 400 mg TKI258, the levels of FGF23 increased up to 1.96-fold and 2.1-fold the basal levels, respectively.

### Example 5

### FGF23 Measurements in Plasma Samples from Melanoma Cancer Patients Treated with TKI258

### 5.1 Methods

Methods: Patients were treated orally with 200, 300, 400 or 500 mg/day on a once daily continuous dose schedule. The MTD was defined at 400 mg/day. Plasma samples from 43 patients were collected. Plasma concentration of TKI258 was measured by LC/MS/MS. Plasma FGF23 was evaluated by ELISA

FGF23 ELISA Assay. To monitor FGF23 plasma samples in patients, the FGF23 ELISA assay from KAINOS Laboratories, Inc., Japan was used (catalogue #CY-4000) as described in previous examples.

### 5.2 Results and Discussion

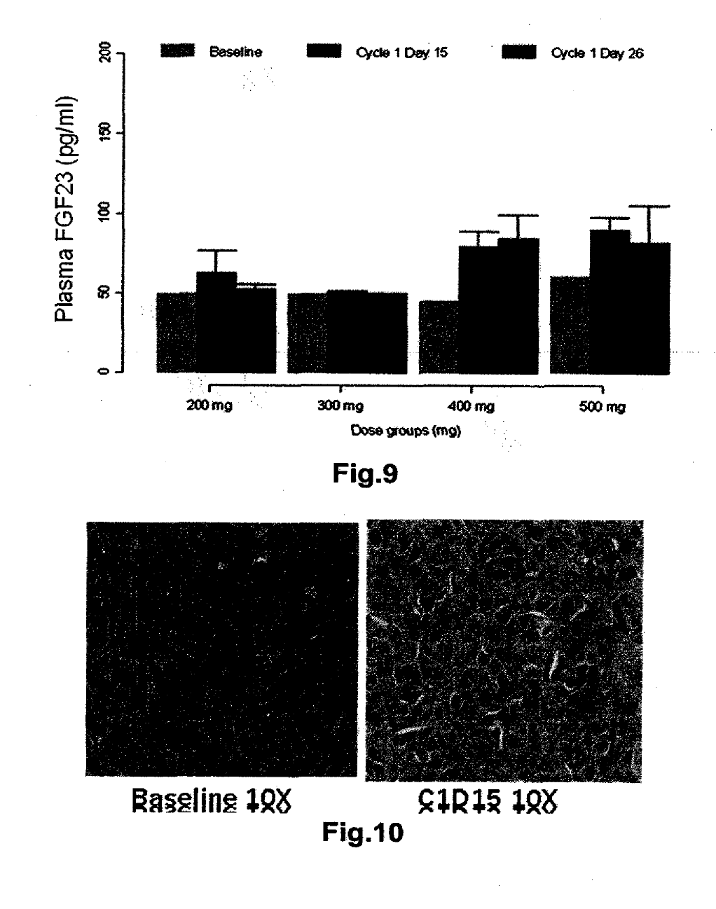

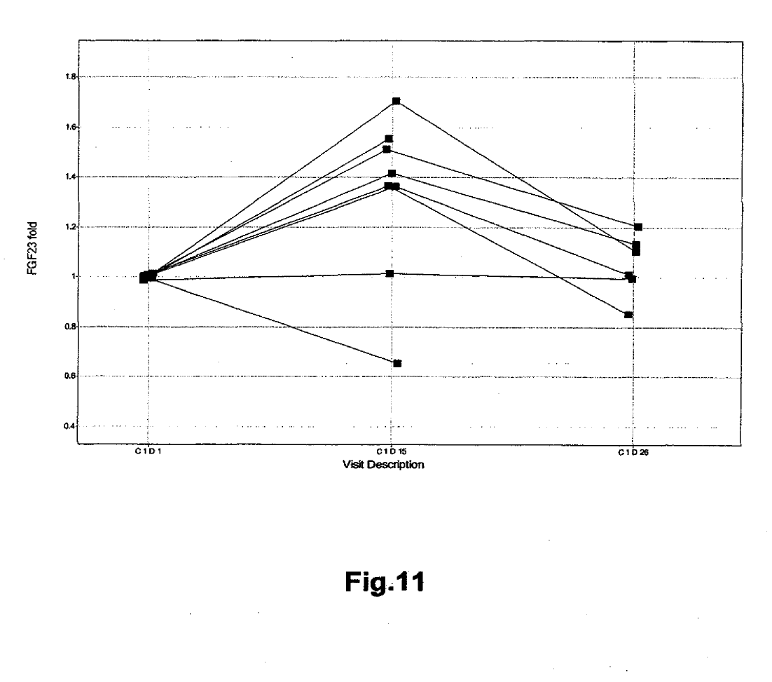

FGF23 data from patients treated with 200 mg, 300 mg, 400 mg or 500 mg daily dose of TKI258 is shown in FIG. 9. Data is presented as the mean of the indicated number of patients. Following 400 mg or 500 mg continuous daily dosing, the mean plasma exposure (AUC24 hr) was approximately 3000 ng/mL*h and 4100 ng/mL*h, respectively. No accumulation in TKI258 plasma exposure was observed at doses of 400 mg or below, while accumulation up to 2.5-fold was observed on day 15 following the 500 mg daily dose. At the end of the first treatment cycle, mean plasma FGF23 levels increased over baseline by 68% while the increase at day 15 of the first treatment cycle is 63%. One patient treated with 400 mg TKI258 showed plasma FGF23 increase over baseline by 98% (from baseline level of 40 pg/ml to about 80 pg/ml) at Cycle 1 Day 15. The tumor biopsy from the same patient at cycle 1 Day 15 showed significant pFGFR inhibition analyzed by immunohistochemistry. (FIG. 10). This result suggests that the induction of plasma FGF23 after TKI258 treatment correlates with FGFR target inhibition in tumor tissues.

Conclusions: Induction of plasma FGF23 suggest that FGFR may be inhibited at doses of 400 mg/day and above.

### Example 6

### FGF23 Measurements in Plasma Samples from Metastatic Renal Cell Carcinoma (mRCC) Patients Treated with TKI258 in a Phase I Clinical Trial

### 6.1 Methods

Patients and treatment: The primary objective of this phase I was to determine the maximum tolerated dose (MTD) of TKI258, administered orally on a 5 days on/2 days off schedule in repeated 28 day cycles, in mRCC pts refractory to standard therapies. A two-parameter Bayesian logistic regression model and safety data for at least 21 pts will be used to determine MTD.

FGF23 ELISA Assay. To monitor FGF23 plasma samples in patients, the FGF23 ELISA assay from KAINOS Laboratories, Inc., Japan was used (catalogue #CY-4000) as described in previous examples.

### 6.2 Results and Discussion

Results: A phase I study is ongoing. As of December 2008, 11 pts (9 m, 2 f), median age: 55 (29-66 yrs) have been enrolled. Four pts have been treated at 500 mg/day (start dose): 2 are ongoing at cycle (C) 7; 1 pt discontinued due to PD and 1 due to sinus bradycardia. Five pts received 600 mg/day: 2 DLTs (G4 hypertension and G3 fatigue—pts discontinued) leading to dose reduction of all patients to 500 mg/day, 2 pts in C5 and C4, 1 pt discontinued for PD. Two pts just entered the extension cohort at 500 mg. Other toxicities ≧G2 included fatigue, nausea, vomiting, diarrhea, neutropenia, folliculitis and dizziness. PK data showed C_{Max} range (180-487 ng/mL, n=8), and AUC range (2200-8251 ng/mL*h). Preliminary biomarker data indicated pts had high baseline VEGF (506: 203 pg/ml, n=6) and bFGF (220: 185 pg/ml, n=6) levels, which may reflect failure of previous anti-VEGF agents. Induction of plasma FGF23 levels, a pharmacodynamic biomarker of FGFR inhibition, was observed in pts from the first 500 mg/day dosing cohort (FGF23 data from individual RCC patients treated with TKI258 is shown in FIG. 11). Preliminary evidence of efficacy is observed with one minor response (−17% at C4), 4 stable disease and 1 dramatic shrinkage/necrosis of some target lesions (lymph node & suprarenal mass).

### Conclusions:

TKI258 500 mg/day seems a feasible schedule in heavily pre-treated mRCC patients with some indications of clinical benefit. Some of the treated patients have clearly increased FGF23 level while of some of the patients do not have that increase. For the patients having increased FGF23, the peak of FGF23 level seemed to be around cycle 1 Day 15. The level of FGF23 has increased in a range of 1.35-1.75 compared to the baseline level.

### Example 7

### FGF23 Induction by TKI258 in Rats Correlates with FGFR3 Inhibition in RT112 Subcutaneous Tumor Xenografts

### 7.1 Methods

Animals. Experiments were performed in female Rowett rats Hsd:RH-Foxlrnu. These athymic Nude-Rats were obtained from Harlan (The Netherlands)

Compound Formulation and Animal Treatment. TK1258 was formulated in acetic acid-acetate buffer (pH 4.6)/PEG300 (2:1 v/v) and applied daily by gavage. Vehicle consisted of acetic acid-acetate buffer (pH 4.6)/PEG300 (2:1 v/v). The application volumes were 5 ml/kg.

Study design: rats were subcutaneously implanted with RT112 xenografts by subcutaneous injection into the right flank of 1×10⁶ RT112 cells in 100 μl HBSS (Sigma #H8264) containing 50% Matrigel (BD #356234). When tumors reached an average volume of 400 mm³, rats received with a single oral administration of TKI258 at 10 mg/kg, 25 mg/kg, or 50 mg/kg or vehicle.

Blood and tissue sampling for ex vivo analysis. Blood samples were drawn sublingually at 3 h, 7 h and 24 h post-compound administration. Plasma and as serum were prepared from each blood sample. At the same time points, tumors were dissected and snap frozen in liquid nitrogen.

Ex vivo analysis of RT112 tumor xenografts: RT112 bladder cancer cells express high levels of FGFR3, the activity of which can be monitored in these cells by measuring changes in FRS2 tyrosine phosphorylation, a substrate of the FGFRs. The tumor material was pulverized using a swing mill (RETSCH, either MM2 or MM200). Aliquots of tumor powder (50 mg) were lysed in ice-chilled lysis buffer containing 50 mM Tris pH 7.5, 150 mM NaCl, 1 mM EGTA, 5 mM EDTA, 1% Triton, 2 mM NaVanadate, 1 mM PMSF and protease inhibitors cocktail Roche #11873580001). Lysates were clarified by centrifugation at 12000×g for 15 min and protein concentration was determined using the DC protein assay reagents (Bio Rad #500-0116) and a BSA standard. Total cell lysates were subjected to SDS-PAGE and proteins blotted onto PVDF membranes. Filters were blocked in 5% BSA and further incubated with the primary antibodies p-FRS2(Tyr196): Cell Signaling #3864; P-tubulin: Sigma #T4026) over-night at 4° C. Proteins were visualized with peroxidase-coupled anti-mouse or anti-rabbit AB using the SuperSignal® West Dura Extended Duration Substrate detection system (Pierce #34075).

FGF23 ELJSA assay. To monitor FGF23 levels in serum samples, the FGF23 ELISA assay from KAINOS Laboratories, Inc., Japan was used (catalogue #CY-4000) as described in previous examples.

### 7.2 Results and Discussion

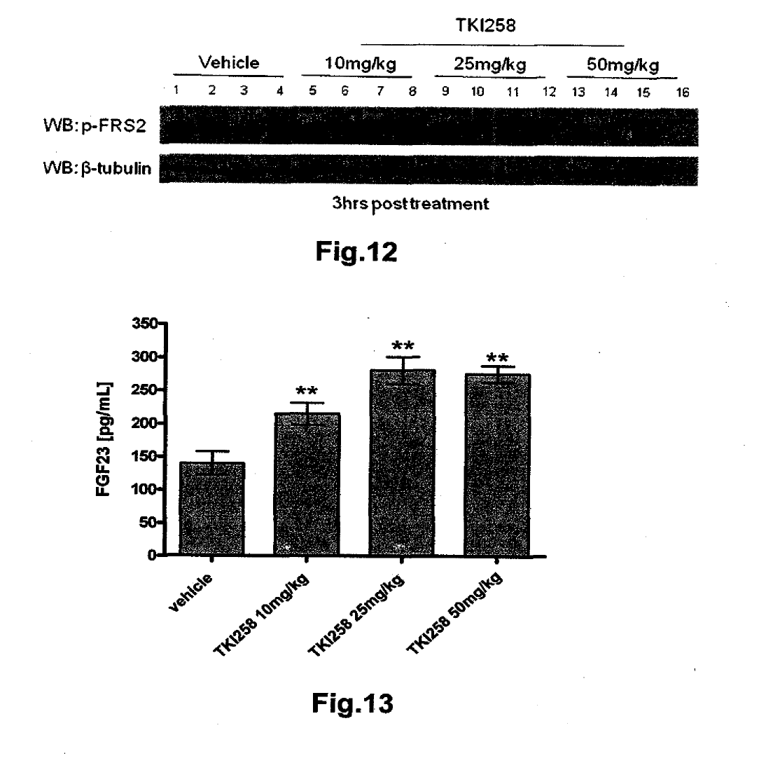

Modulation of FRS2 tyrosine phosphorylation in RT112 xenografts upon administration of TK1258 to rats. FRS2 is a substrate of the FGFRs that is phosphorylated on tyrosine residues by activated FGFRs and thus can be used as a read-out for FGFR activity. Analysis of RT112 tumors from animals treated with 10, 25 or 50 mg/kg TKI258 or vehicle, dissected at 3 h post-treatment showed that TKI258 inhibited FRS2 tyrosine phosphorylation in a dose-dependent manner (FIG. 12).

FGF23 levels in serum samples of Rowett rats FGF23 levels were determined in serum samples from rats treated with TKI258 or vehicle, 24 hours post dosing. Rats that were treated with TKI258 showed a dose-dependent increased in serum levels of FGF23 as compared to the vehicle-treated group (FIG. 13), which was statistically significant. (p<0.01, ANOVA post hoc Dunnett's). Data are presented as means±SEM.

Conclusion. The experimental data presented demonstrates that doses of TKI258 that inhibit FGFR3 in vivo, as determined by inhibition of FRS2 tyrosine phosphorylation, also lead to increased levels of serum FGF23 in a dose dependent manner.

### Example 8

### FOF23 Induction by PD173074 in Rats and Comparison to COMPOUND A and TKI258

### 8.1 Methods

Animals. Experiments were performed in female wistar rats furth WF/Ico

Compounds, Formulation and Animal treatment.

PD173074, COMPOUND A and TKI258 were formulated as solutions in NMP (1-Metyl-2-pyrrolidone)/PEG300 1:9 (1 ml NMP+9 ml PEG300) and applied daily by gavage. The application volumes were 5 ml/kg.

Study design rats were treated with a single oral administration of PD173074 (50 mg/kg), COMPOUND A (10 mg/kg) or TKI258 (50 mg/kg) at or vehicle.

Blood and tissue sampling for ex vivo analysis. Blood samples were drawn sublingually at 24 h post-compound administration. Plasma, as well as serum samples were prepared from each blood sample.

FGF23 ELISA assay. To monitor FGF23 levels in serum samples, the FGF23 ELISA assay from KAINOS Laboratories, Inc., Japan was used (catalogue #CY-4000) as indicated in previous examples.

### 8.2 Results and Discussion

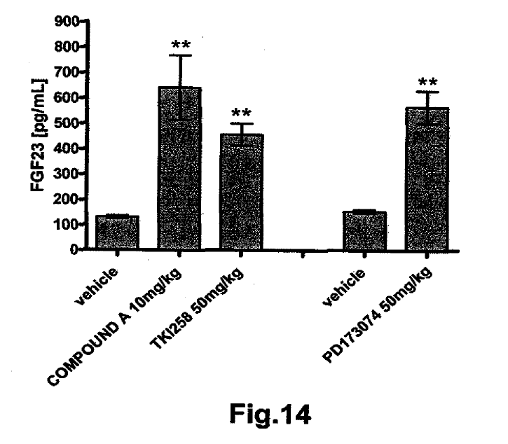

FGF23 levels in serum samples of wister rats. Rats that were treated with PD173074 or COMPOUND A or TKI258 showed a statistically significant increased in serum levels of FGF23 as compared to the vehicle-treated group (FIG. 14). (p<0.01, ANOVA post hoc Dunnett's). Data are presented as means±SEM.

Conclusion. The experimental data presented demonstrates that the FGFR inhibitors PD173074, COMPOUND A or TKI258 cause an increase in serum levels FGF23 in rats.

1. A method of selectively treating a proliferative disease, comprising:
a) selecting a patient for treatment with an FGFR inhibitor on the basis of said patient having an elevated level of inorganic phosphorous; and
b) selectively administering a therapeutically effective amount of a FGFR inhibitor to the patient.

2. The method of claim 1, wherein the FGFR inhibitor is selected from the group consisting of compound A (3-(2,6-Dichloro-3,5-dimethoxy-phenyl)-1-{6-[4-(4-ethyl-piperazin-1-yl)-phenylamino]-pyrimidin-4-yl}-1-methyl urea), or any pharmaceutical acceptable salts thereof; and TKI258, or any pharmaceutical acceptable salts thereof.

3. A method for determining the inhibition of kinase activity of fibroblast growth factor receptor (FGFR), comprising the steps of administering a therapeutically effective amount of a FGFR inhibitor to a subject, assaying a biological sample of said subject for the level of inorganic phosphorus (P) of said biological sample, and comparing said level of inorganic phosphorus (P) of said biological sample with a reference level.

4. A method for determining one or more secondary effects of a FGFR inhibitor comprising steps a) to d) of claim 2, further comprising the steps of
e) correlating said level of inorganic phosphorus (P) with one or more secondary effects; and
f) determining said level of inorganic phosphorus (P) above which secondary effect occur relatively to the treatment employed.

5. The method of claim 4, wherein the secondary effect is ectopic mineralization.

6. The method of claim 4, wherein the level of inorganic phosphorus (P) is increased when compared to the reference level.

7. An ex vivo method for determining the modulation of kinase activity of FGFR comprising the steps of
a) determining inorganic phosphorus (P) level in a biological sample of a patient before the onset of a FGFR inhibitor treatment (individual reference level);
b) determining inorganic phosphorus (P) level in a biological sample of the same patient after said FGFR inhibitor treatment wherein the increased inorganic phosphorus (P) level of step b) over the individual reference level indicates the inhibition, of the kinase activity of FGFR occurred.

8. The method of claim 7, wherein said FGFR inhibitor selected from the group consisting of compound A (3-(2,6-Dichloro-3,5-dimethoxy-phenyl)-1-{6-[4-(4-ethyl-piperazin-1-yl)-phenylamino]-pyrimidin-4-yl}-1-methyl urea), or any of pharmaceutical acceptable salts thereof; and TKI258, or any of pharmaceutical acceptable salts thereof.

9. The method of claim 7, wherein said FGFR inhibitor is compound A, or any of pharmaceutical acceptable salts thereof.

10. A kit for use in treating a patient having a proliferative disease comprising,
a) a therapeutically effective amount of a therapeutically effective amount of a FGFR inhibitor,
b) at least one probe capable of detecting the presence of inorganic phosphorous;
c) instructions for using the probe to assay a biological sample from the patient for the presence of the inorganic phosphorous,
d) instructions for administering the therapeutically effective amount of a FGFR inhibitor to the patient if the biological sample from the patient has an elevated amount of the inorganic phosphorous; and
e) optionally, means for administering the therapeutically effective amount of a FGFR inhibitor t to the patient.
